SEQUENCE LISTING (6 sequences)

SEQ ID NO: 1 - DNA, 422 bp, Human
ggctgccgaagatggcggaggtgcaggtcctggtgctcgatggtcgaggccatctcctggtccgcctggcggccatcgtggctaaacaggtactgctgggccggaaagtggtggtcgtacgctgcgaaggcatcaacatttctggcaatttctacagaaacaagttgaagtacctgggtttcctccgcaagcggatgaacacccacctttcccgaggtccctaccacttccgggccccccagccgcatcttctggcggaccgtgcgaggtatgccgccccacaagaccaagcgaggccaggcttctctggaccgcctcaaggtgtttgaccgcatcccaccgccctacgacaagaaaaagcggatgctggaagtaccaggcagtgacagccaccctggaggagaagaggaaagagaaagcca

SEQ ID NO: 2 - DNA, 1470 bp, Human
tattctcttagcttgtgttggccaattgtttgcttatgggggaatgacttttgaagacttgatctagagatggaatccacagtcctctttctcatttcatccaaactgagtctgctgttttgtgttttatttatagagcagtcaggttcctttcttccctgaagccaacctagtacctagggcactaagattatgttaagaggcttttgtgtgctaatgtgctaattcaaggctgatggaagtgaatttttatcataataatgtgaataaaatacatttttctgaaaaaaaaaagtgagttctcaccaaaaccagtggaaggagcccatgatccaccaaacagggacttctcagctacaaatgggaacgtttgtgtctccagctgggctgcagctccacctgcagaatgaggaggaagggaccacaaagtaaacaggtgatagtcattactaacatttccatcatctgcttttcctctcaatggccagttaacacaagatgtcctcttgcacagatgcagaatctcataagccatcaactttaccctgaatagaagtaaaaaggtctttattcatttttcctcccccctaaatttattaaatacctgatagatgtcaaacactgttaggtatgaagatacagtcatgagtgaagcatgttcttggaaagaagacatagcccagctctccatagaaatgaaatacagcaataatatatgtatttataataggttaatgggtttttttgtctacaaaaaaaaacaaatttttctatcacttagcaaagtgactaggtcattttacttttttgaacttgattatttggctaatattataaaatgccagagctaaaaatagctgtacctggggtgaaatggagaagacgtgggacatagctttaaaaatgggagaagcgctttttcccaagcggctgccgaagatggcggaggtgcaggtcctggtgctcgatggtcgaggccatctcctggtccgcctggcggccatcgtggctaaacaggtactgctgggccggaaagtggtggtcgtacgctgcgaaggcatcaacatttctggcaatttctacagaaacaagttgaagtacctgggtttcctccgcaagcggatgaacacccacctttcccgaggtccctaccacttccgggccccccagccgcatcttctggcggaccgtgcgaggtatgccgccccacaagaccaagcgaggccaggcttctctggaccgcctcaaggtgtttgaccgcatcccaccgccctacgacaagaaaaagcggatggtgttcctgctccctcaaggttgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctac

SEQ ID NO: 3 - DNA, 60 bp, Human
taagccatcaactttaccctgaatagaagtaaaaaggtctttattcatttttcctccccc

SEQ ID NO: 4 - DNA, 600 bp, Human
ggacatagctttaaaaatgggagaagcgctttttcccaagcggctgccgaagatggcggaggtgcaggtcctggtgctcgatggtcgaggccatctcctggtccgcctggcggccatcgtggctaaacaggtactgctgggccggaaagtggtggtcgtacgctgcgaaggcatcaacatttctggcaatttctacagaaacaagttgaagtacctgggtttcctccgcaagcggatgaacacccacctttcccgaggtccctaccacttccgggccccccagccgcatcttctggcggaccgtgcgaggtatgccgccccacaagaccaagcgaggccaggcttctctggaccgcctcaaggtgtttgaccgcatcccaccgccctacgacaagaaaaagcggatggtgttcctgctccctcaaggttgtgcgtctgaagcctacaagaaagtttgcctatctggggcgcctggctcacgaggttggctggaagtaccaggcagtgacagccaccctggaggagaagaggaaagagaaagccaagatccactaccggaagaagaaacagctcatgaggctacggaaacaggccgagaag

SEQ ID NO: 5 - DNA, 366 bp, Human
ggctgccgaagatggcggaggtgcaggtcctggtgctcgatggtcgaggccatctcctggtccgcctggcggccatcgtggctaaacaggtactgctgggccggaaagtggtggtcgtacgctgcgaaggcatcaacatttctggcaatttctacagaaacaagttgaagtacctgggtttcctccgcaagcggatgaacacccacctttcccgaggtccctaccacttccgggccccccagccgcatcttctggcggaccgtgcgaggtatgccgccccacaagaccaagcgaggccaggcttctctggaccgcctcaaggtgtttgaccgcatcccaccgccctacgacaagaaaaagcggatg

SEQ ID NO: 6 - DNA, 56 bp, Human
ctggaagtaccaggcagtgacagccaccctggaggagaagaggaaagagaaagcca

(19) United States
(12) Patent Application Publication
(10) Pub. No.: US 2004/0219560 A1
(43) Pub. Date: Nov. 4, 2004

(54) Method, program and apparatus for predicting gene expression

(75) Inventor: Itaru Sakou, Kawasaki (JP)

Correspondence Address: STAAS & HALSEY LLP, SUITE 700, 1201 NEW YORK AVENUE, N.W., WASHINGTON, DC, 20005, US

(73) Assignee: FUJITSU LIMITED, Kawasaki (JP)

(21) Appl. No.: 10/629,897

(22) Filed: Jul. 30, 2003

(30) Foreign Application Priority Data
Jul. 31, 2002 (JP) 2002-224075

(51) Int. Cl. C12Q 1/68

(52) U.S. Cl. 435/6

(57) ABSTRACT

A method for predicting an expression sites of an unknown gene includes: inputting sequence of the unknown gene; mapping the unknown gene onto a genome sequence; calculating a distance on the genome sequence between a known gene and the unknown; and extracting an expression sites of the unknown gene based on the calculated distance.

### BACKGROUND OF THE INVENTION

1) Field of the Invention

The present invention relates to a method, program and apparatus for predicting gene expression sites of a gene whose expression site is unknown on a genome sequence.

2) Description of the Related Art

While recent progress on the genetic engineering facilitates decoding of gene sequences, as the next approach, the functions of unknown genes on the genome sequences are analyzed. On analyzing genetic functions, prediction of the functions based on gene expression sites is effective. Therefore, a technique for predicting an expression site of unknown genes is required.

For predicting expression sites of an unknown gene, there are two methods. The first method is practically and experimentally exploring a tissue with expression site of the unknown gene. The second method is homology searching a large number of expressed sequence tag (hereinafter, “EST”) sequences for unknown gene sequences, using a computer. This second method includes searching an EST database for a homology of gene sequences to be predicted and extracting expression information on the EST from the result of searching.

The first method has a problem in that functional analysis is not carried out efficiently because it depends on experiments. The second method has a problem in that fast functional analysis is prevented because it takes a longer time for management of a large number of EST sequences and the homology searching.

### SUMMARY OF THE INVENTION

It is an object of the present invention to at least solve the problems in the conventional technology.

The method for predicting gene expression sites according to one aspect of the present invention includes calculating a distance between first and second genes on a genome sequence, wherein an expression site of the first gene is unknown, and the second gene is one of a plurality of genes whose expression sites are known; and determining the expression sites of the first gene based on the distance.

The computer program product according to another aspect of the present invention realizes the method according to the present invention on a computer.

The apparatus for predicting gene expression sites according to still another aspect of the present invention includes a calculation unit that calculates a distance between first and second genes on a genome sequence, wherein an expression site of the first gene is unknown, and the second gene is one of a plurality of genes whose expression sites are known; and a determination unit that determines the expression sites of the first gene based on the distance.

The other objects, features and advantages of the present invention are specifically set forth in or will become apparent from the following detailed descriptions of the invention when read in conjunction with the accompanying drawings.

### BRIEF DESCRIPTION OF THE DRAWINGS

FIG. 1 is an example of arrangement of known genes and an unknown gene on a genome sequence;

FIG. 2 is a graph of the number of gene pairs having the same expression site versus distance between genes;

FIG. 3 is a hardware block diagram of an apparatus for predicting gene expression sites according to the embodiment of the present invention;

FIG. 4 is a functional block diagram of the apparatus for predicting gene expression sites;

FIG. 5 is a flowchart of procedures performed by the apparatus for predicting gene expression sites;

FIG. 6 is an example of sequence information on a sequence of an unknown gene;

FIG. 7 is an example of sequence information on a sequence of a known genome;

FIG. 8 is an example of comparison between the sequences of the known genome and the unknown gene;

FIG. 9 is an example of name and positional information of known genes;

FIG. 10 is an example of computational result of distance between a known gene and an unknown gene;

FIG. 11 is an example of an expression profile;

FIG. 12 is an example of expression information of known genes;

FIG. 13 is another example of expression information of known genes;

FIG. 14 is a diagram showing a positional relation of genes on a genome sequence, from which the sensitivity and the specificity are derived;

FIG. 15 is a diagram (graph) showing computational results using the known data associated with the human chromosome 19;

FIG. 16 is a diagram (graph) showing computational results using the known data associated with the human chromosome 21;

FIG. 17 is a diagram showing the predicted results of the expression sites of the known gene (ABCC13);

FIG. 18 is a first diagram showing the predicted results of the expression sites of another known gene (Human gene GPR40); and

FIG. 19 is a second diagram showing the predicted results of the expression sites of another known gene (Human gene GPR40).

### DETAILED DESCRIPTION

Exemplary embodiments of a method, a computer program product, and an apparatus relating to the present invention will be explained in detail below with reference to the accompanying drawings.

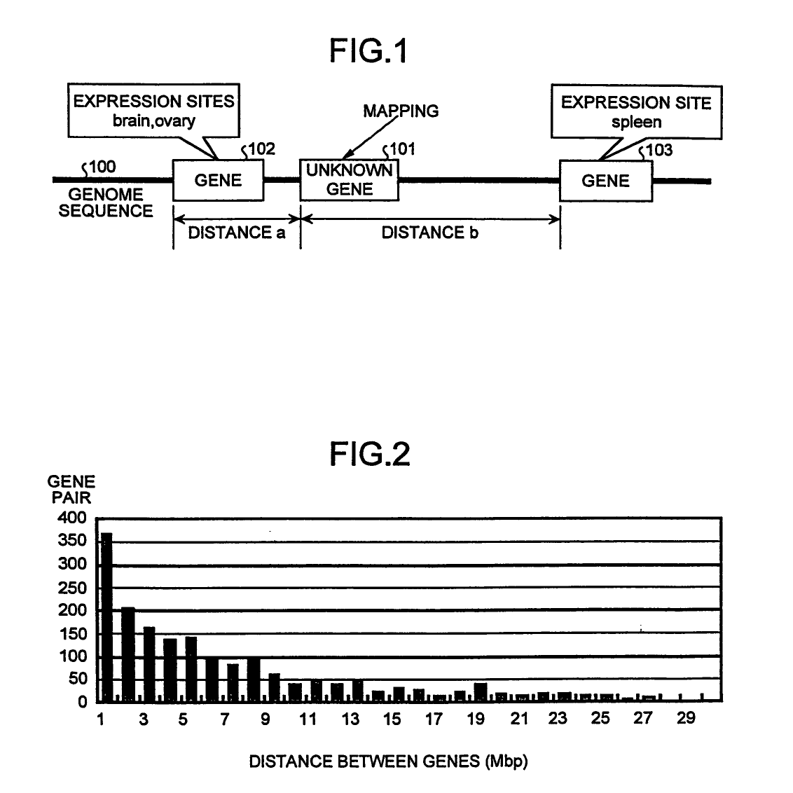

FIG. 1 is an example of arrangement of known genes and an unknown gene on a genome sequence. An unknown gene 101 is mapped on the genome sequence 100. Known genes 102 and 103, which are located around the unknown gene 101, are specified on the genome sequence 100. The known gene (hereinafter, “surrounding gene”) 102 and the surrounding gene 103 are scored by distances (distances “a”, “b”) from the unknown gene 101. These distances are employed in prediction of expression sites of the unknown gene 101.

The surrounding gene 102 has expression sites in brain and ovary and has a distance “a” from the unknown gene 101. The surrounding gene 103 has an expression site in spleen and has a distance “b” from the unknown gene 101. As the distance “a” is less than the distance “b”, it can be predicted that the expression sites of the unknown gene 101 is more relevant to brain or ovary.

FIG. 2 is a graph of the number of gene pairs (human chromosome 21) having the same expression site versus distance (million base-pair) between genes. As can be seen from the graph, genes on the genome sequence 100 spaced by shorter distances from each other exhibit a trend to express on the same tissue. The method according to the embodiment is for predicting the expression sites of the unknown gene 101 based on this trend.

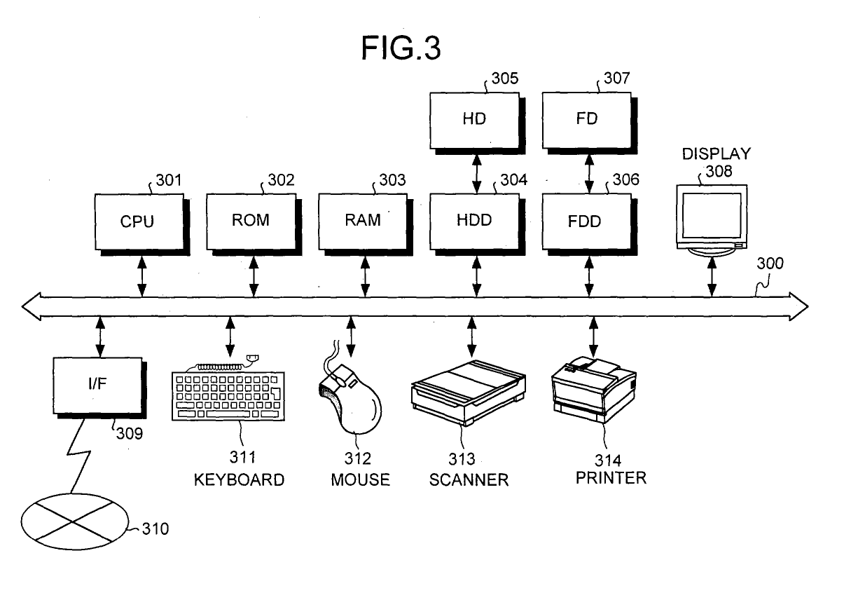

FIG. 3 is a hardware block diagram of an apparatus for predicting gene expression sites (hereinafter, “expression predicting apparatus”) according to the embodiment of the present invention. The expression predicting apparatus shown in FIG. 3 includes a central processing unit (hereinafter, “CPU”) 301, a read only memory (hereinafter, “ROM”) 302, a random access memory (hereinafter, “RAM”) 303, a hard disk dive (hereinafter “HDD”) 304, a hard disk (hereinafter, “HD”) 305, a flexible disk drive (hereinafter, “FDD”) 306, a flexible disk (hereinafter “FD”) 307 as an exemplary detachable storage medium, a display 308, a communication interface (hereinafter, “I/F”) 309, a keyboard 311, a mouse 312, a scanner 313, and a printer 314. These elements are connected to each other via a bus 300.

The CPU 301 controls the expression predicting apparatus. The ROM 302 stores programs such as a boot program. The RAM 303 is employed as a work area for the CPU 301. The HDD 304 controls read/write of data from/to the HD 305 under the control of the CPU 301. The HD 305 stores data written under the control of the HDD 304.

The FDD 306 controls read/write of data from/to the FD 307 under the control of the CPU 301. The FD 307 stores data written under the control of the FDD 306 and allows the expression predicting apparatus to read out the data stored in the FD 307. Other detachable storage media than the FD 307 may include a CD-ROM (CD-R, CD-RW), a magneto-optical disk (hereinafter “MO”), a digital versatile disk (hereinafter “DVD”) and a memory card. The display 308 displays a cursor, icons, toolboxes, and data such as documents, images, and functional information. The display 308 is, for example, a cathode ray tube (CRT), a thin film transistor (TFT)-liquid crystal display or a plasma display.

The I/F 309 is connected to a network 31 such as a local area network (LAN) or the Internet via a communication channel, and further connected via the network 310 to network nodes or server machines equipped with databases. The I/F 309 interfaces between the internal of the expression predicting apparatus and the network 310 to control input/output of data from/to the servers and the network nodes. The I/F 309 is, for example, a modem.

The keyboard 311 has a plurality of keys for entering characters, numerals and various instructions to input data. The keyboard 311 may be input pads of a touch panel type or a ten-key. The mouse 312 is employed to move the cursor, select a range to be processed, and move and resize a window. Pointing devices such as a track ball and a joystick may be employed, instead of the mouse 312.

The scanner 313 optically reads images such as graphics and pictures and sends them as image data to the expression predicting apparatus. The scanner 313 also has an optical character reader (hereinafter, “OCR”) function to obtain data indicating contents of a document from printed one. The printer 314 prints image data and document data, and is, for example, a laser printer or an inkjet printer.

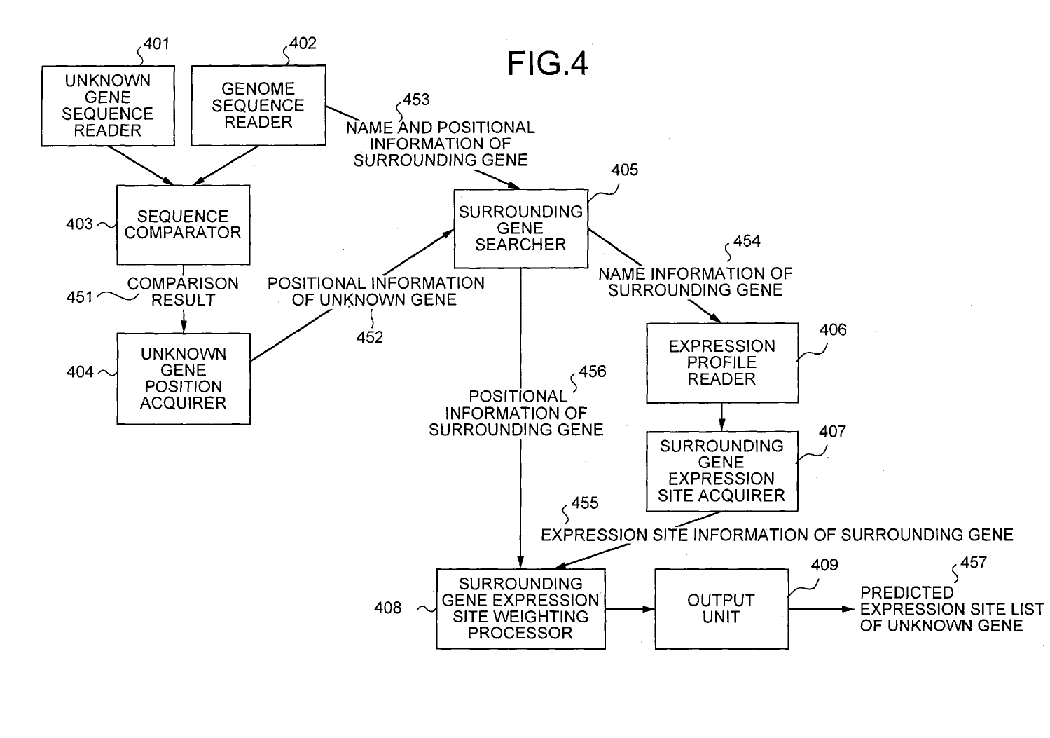

FIG. 4 is a functional block diagram of the expression predicting apparatus. An unknown gene sequence reader 401 reads sequence information of the unknown gene 101. The function of the unknown gene sequence reader 401 can be achieved using the I/F 309 or the keyboard 311, the mouse 312 and the scanner 313.

The genome sequence reader 402 reads information about the genome sequence 100 associated with the unknown gene 101. The function of the gene sequence reader 402 can also be achieved using the I/F 309 or the keyboard 311, the mouse 312 and the scanner 313 similar to the unknown gene sequence reader 401.

The sequence comparator 403 compares the sequence information on the gene sequence 101 read by the unknown gene sequence reader 401 with the information on the genome sequence 100 read by the genome sequence reader 402. From the result of comparing, the sequence comparator 403 maps the sequence information on the unknown gene 101 onto the genome sequence 100. This mapping is detailed later.

The unknown gene position acquirer 404 acquires a comparison result 451 obtained from the sequence comparator 403, that is, a position on the genome sequence 100 of the unknown gene 100 mapped on the genome sequence 100. The acquired position is sent to the surrounding gene searcher 405 as positional information of unknown gene 452.

The surrounding gene searcher 405 searches for surrounding genes (genes 102, 103) having specified expression sites and located around the position of the unknown gene 101 on the genome sequence 100, based on name and positional information of surrounding gene 453 received from the genome sequence reader 402. As a result, the surrounding gene searcher 405 sends name information of surrounding gene 454 to the expression profile reader 406 and positional information of surrounding gene 456 to the surrounding gene expression site weighting processor 408.

From an expression profile database 553 later described, the expression profile reader 406 reads, under the control of the surrounding gene searcher 405, an expression profile corresponding to the surrounding gene name information.

From the expression profile read by the expression profile reader 406, the surrounding gene expression site acquirer 407 acquires the information on the expression sites specified by the surrounding gene and sends it as expression information of surrounding gene 455 to the surrounding gene expression site weighting processor 408.

The surrounding gene expression site weighting processor 408 computes distances on the genome sequence 100 between the surrounding genes 102, 103 having specified expression sites and a position of the unknown gene 101 to specify an expression site of the unknown gene 101 based on the computed distances. For example, the expression site(s) of one of more surrounding gene having shorter computed distances can be specified as the expression sites of the unknown gene 101.

The processor 408 computes distances between the surrounding genes having specified expression sites on the genome sequence information and a position of the unknown gene on the genome sequence. The processor 408 then sorts the surrounding genes in ascending order of computed distance. If a surrounding gene has the same expression information as that of the preceding surrounding gene in the sorted genes, the information is merged. In other words, the information about the same expression site is deleted to allow other information than the information about the expression site to remain. An output unit 409 outputs only the remaining information other than the information about the same expression site.

As for the sequence comparator 403, the unknown gene position acquirer 404, the surrounding gene searcher 405, the surrounding gene expression site acquirer 407, and the surrounding gene expression site weighting processor 408, their functions can be achieved when the CPU 301 executes a program stored in the ROM 302, the RAM 303, the HD 305 or the FD 307 shown in FIG. 3.

The output unit 409 outputs the information about the expression sites processed by the surrounding gene expression site weighting processor 408 in a sorted-order of surrounding genes. The acquired information about the expression sites may be shown in a list, which corresponds to a predicted expression site list of unknown gene 457. The output unit 409 may allow the FD 307 and the I/F 309 shown in FIG. 3, to output the information to external, for example. It may also allow the printer 314 to print the information and the display 308 to display it.

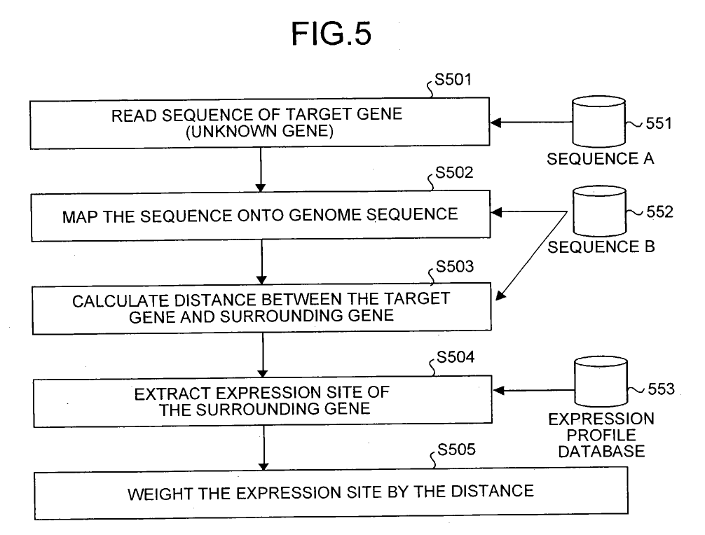

FIG. 5 is a flowchart of procedures performed by the gene expression predicting apparatus. First, the gene expression predicting apparatus reads information of a sequence of an unknown gene to be predicted from a database 551 (Step S501). The unknown gene is hereinafter referred to as “target gene” and the sequence of the target gene as “sequence A”. Next, information of genome sequence (hereinafter, “sequence B”) is read from a database 552 and then is mapped the sequence A onto the sequence B (Step S502). Further, the gene expression predicting apparatus calculates a distance between the target gene and a surrounding gene located around the target gene (Step S503). After that, an expression site of the surrounding gene is extracted from the expression profile database 553 (Step S504). Finally, the gene expression predicting apparatus weights the expression sites of the surrounding gene by the distance and outputs the weighted expression sites (Step S505).

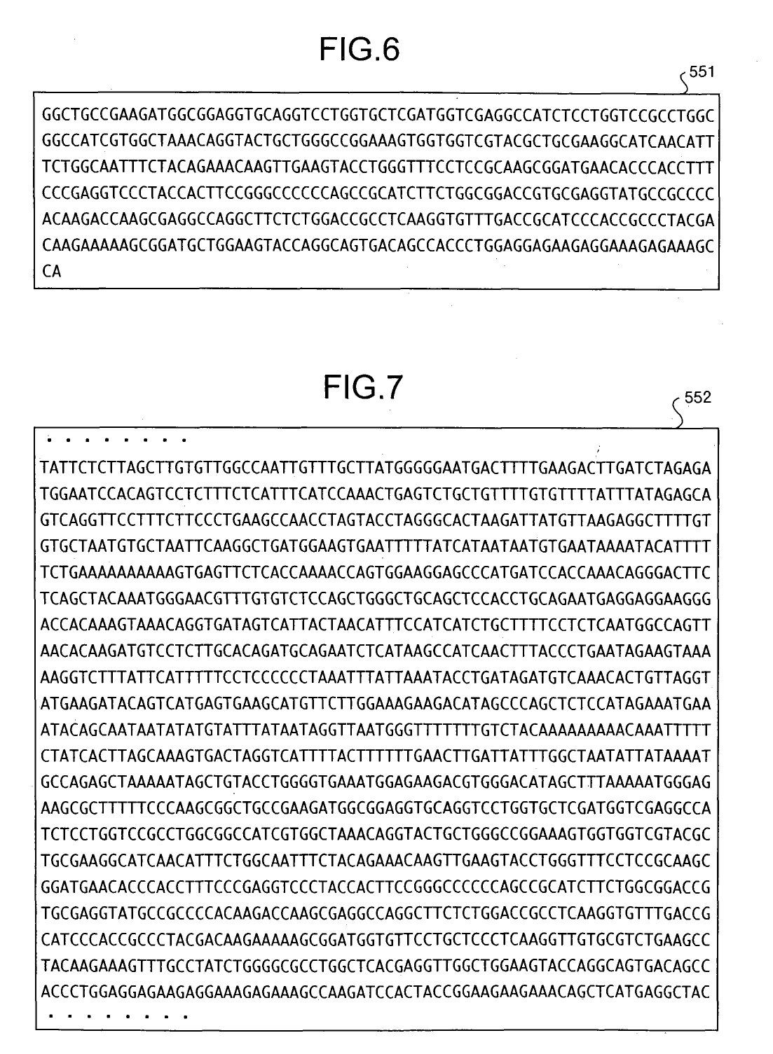

These steps will be explained below in detail. FIG. 6 is an example of information of the sequence A read at Step S501. The information of the sequence A may be entered via the network 310 or a storage medium. In another method, it may be entered directly using the keyboard 311. In an alternative method, the scanner 313 having an OCR function may be employed to enter the information of the sequence A as image information, which is then converted into text data.

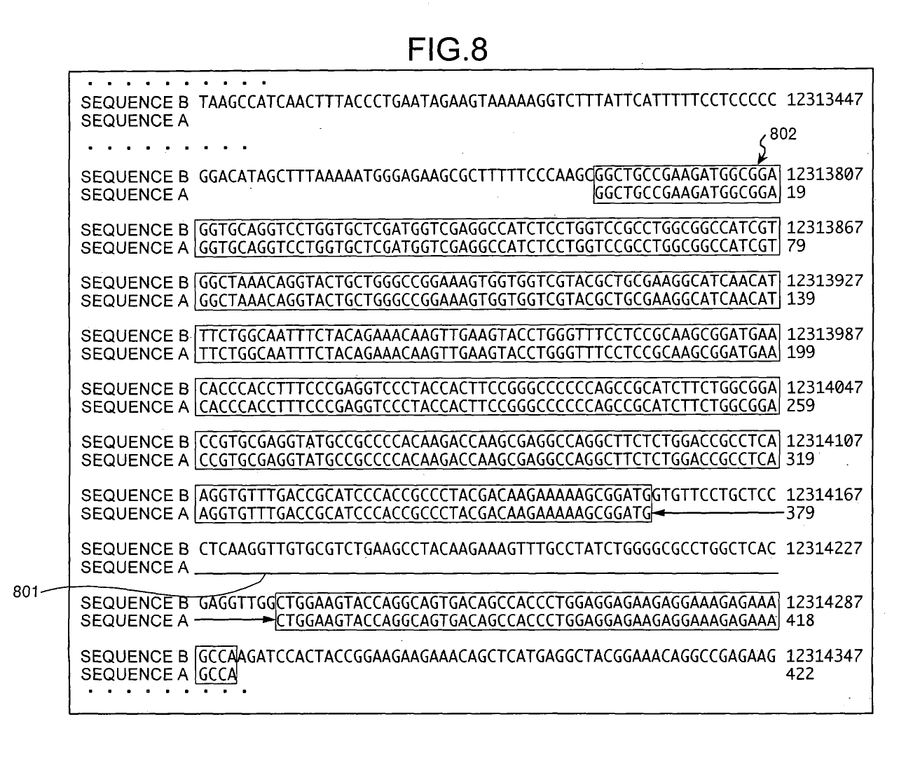

FIG. 7 is an example of information of the sequence B onto which the sequence A is mapped at Step S502. FIG. 8 is an example of comparison between the sequence B and the sequence A, and the comparison indicates the result of homology searching of the sequence A, that is, similar regions in the sequence B shown in FIG. 7. In other words, the comparison shown in FIG. 8 indicates the result of mapping at Step S502. The relation between the sequence A and the sequence B are required as follows: 1) the sequence A is mostly mapped onto the sequence B; 2) a gap 801 may be present in the sequence A which has been mapped onto the sequence B; and 3) if the sequence A has the gap 801, each fragment of the sequence A divided by the gap 801 is mapped onto the sequence B in the order of appearance.

As can be seen from FIG. 8, a position (start position) of the sequence A, which is denoted as the reference number 802, on the sequence B is “12313789” (base-pair).

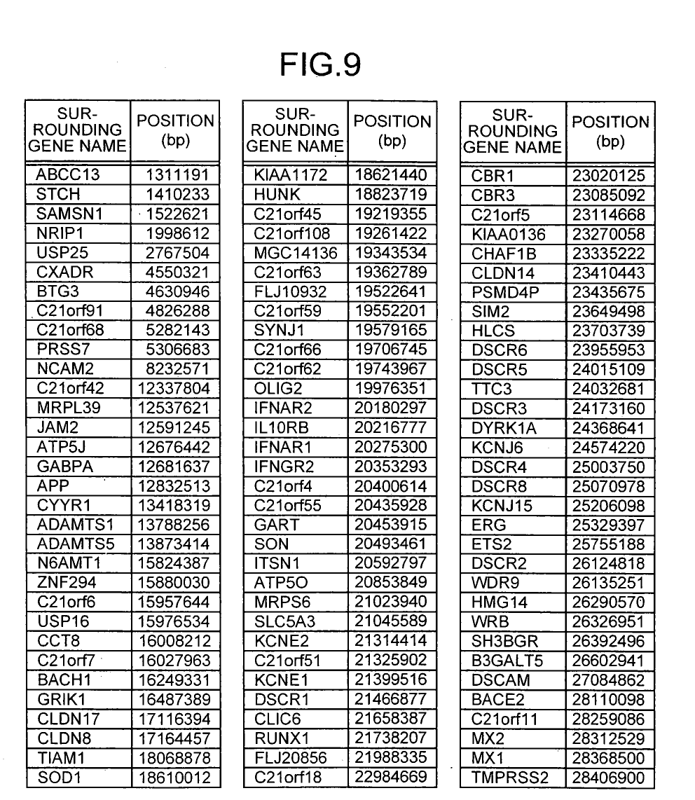

FIG. 9 is an example of name and positional information of surrounding genes around the sequence A, and the information is employed for calculating at Step S503. In FIG. 9, names of surrounding genes are described on the left column and positions (start positions) of the surrounding genes on the right column. The surrounding genes described are sorted in the order of appearance on the genome sequence 100. They may be sorted at the time of output in the step S505.

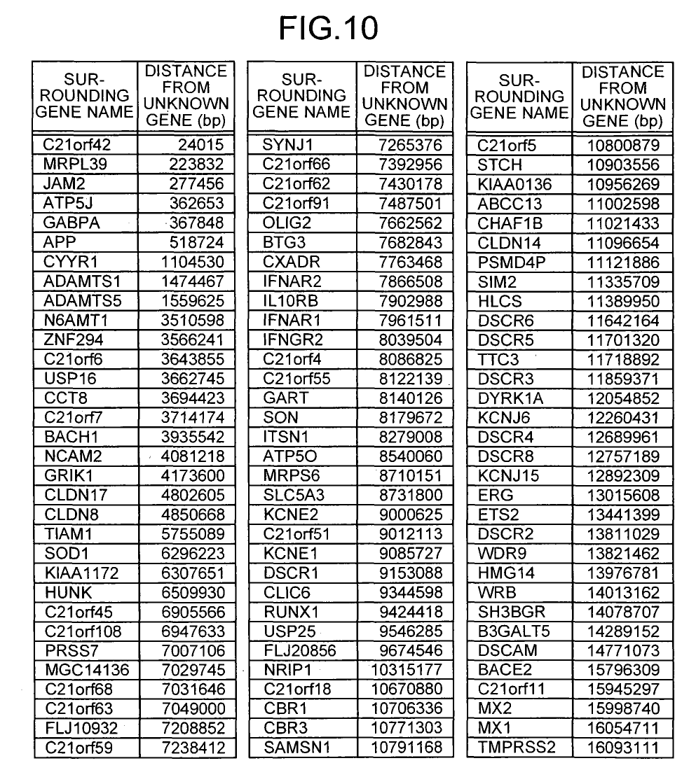

FIG. 10 is an example of computational results of distances between the sequence A and the surrounding genes, which are calculated by the following equation:

Distance (bp) between Sequence A of target gene and

Surrounding gene=|(Position (bp) of Sequence A on Sequence B)−(Position (bp) of Surrounding gene on Sequence B)|

In FIG. 10, names of surrounding genes are described on the left column and distances of the surrounding genes from the target gene on the right column. The surrounding genes described are sorted in ascending order of the distance. For example, as can be seen from FIG. 9, the position of a surrounding gene “C21orf42” on the sequence B is “12337804”(bp), and the position of the sequence A of the target gene on the sequence B is “12313789” (bp). Therefore, for this example, the distance from the target gene is “| 72313789−12337804|=“24015” (bp). For another example, as can be seen from FIG. 9, since a surrounding gene “ADAMTS 1” has a position on the sequence B at “13788256” (bp), its distance from the target gene is “| 12313789−13788256|=“1474467” (bp).

As a result, returning to FIG. 1, the distance between the surrounding gene 102 or 103 whose expression sites are known and the unknown gene 101 is calculated based on the start positions of both genes on the genome sequence 100. However, the start point of the distance may be a position other than the start position. For example, the distance between the both genes on the genome sequence 100 may be calculated as follows: 1) a distance between the end position of the unknown gene 101 and the end position of the surrounding gene; 2) a distance between the start position of the unknown gene 101 and the end position of the surrounding gene, without depending on the order of the surrounding gene and the unknown gene 101 on the genome sequence 100; 3) a distance between the end position of the unknown gene 101 and the start position of the surrounding gene, without depending on the order of the surrounding gene and the unknown gene 101 on the genome sequence 100; 4) a distance between any middle position (e.g. center) between the start and end positions of the unknown gene 101 and any middle position (e.g. center) between the start and end positions of the surrounding gene; 5) a distance between any middle position (e.g. center) between the start and end positions of the unknown gene 101 and the start position of the surrounding gene; 6) a distance between any middle position (e.g. center) between the start and end positions of the unknown gene 101 and the end position of the surrounding gene; 7) a distance between the start position of the unknown gene 101 and any middle position (e.g. center) between the start and end positions of the surrounding gene; and 8) a distance between the end position of the unknown gene 101 and any middle position (e.g. center) between the start and end positions of the surrounding gene.

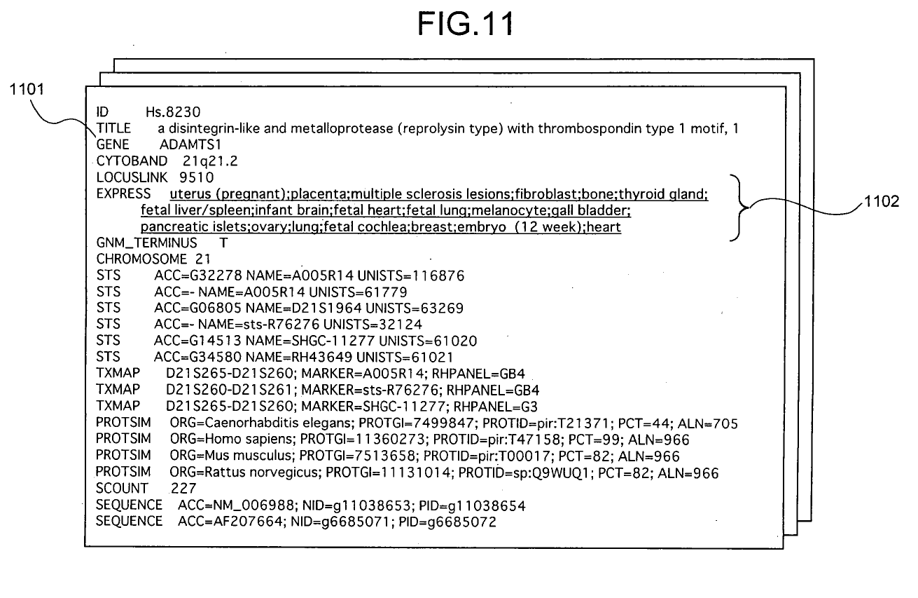

FIG. 11 is an example of an expression profile employed for extracting at Step S504, and the expression profile indicates contents of a surrounding gene “ADAMTS 1”. In the item field “GENE” denoted by reference number 1101, an abbreviated name of the gene is recorded. In the item field “EXPRESS” denoted by reference number 1102, information about the expression sites are recorded. Other items are omitted to explain because they are not directly relevant to the embodiment.

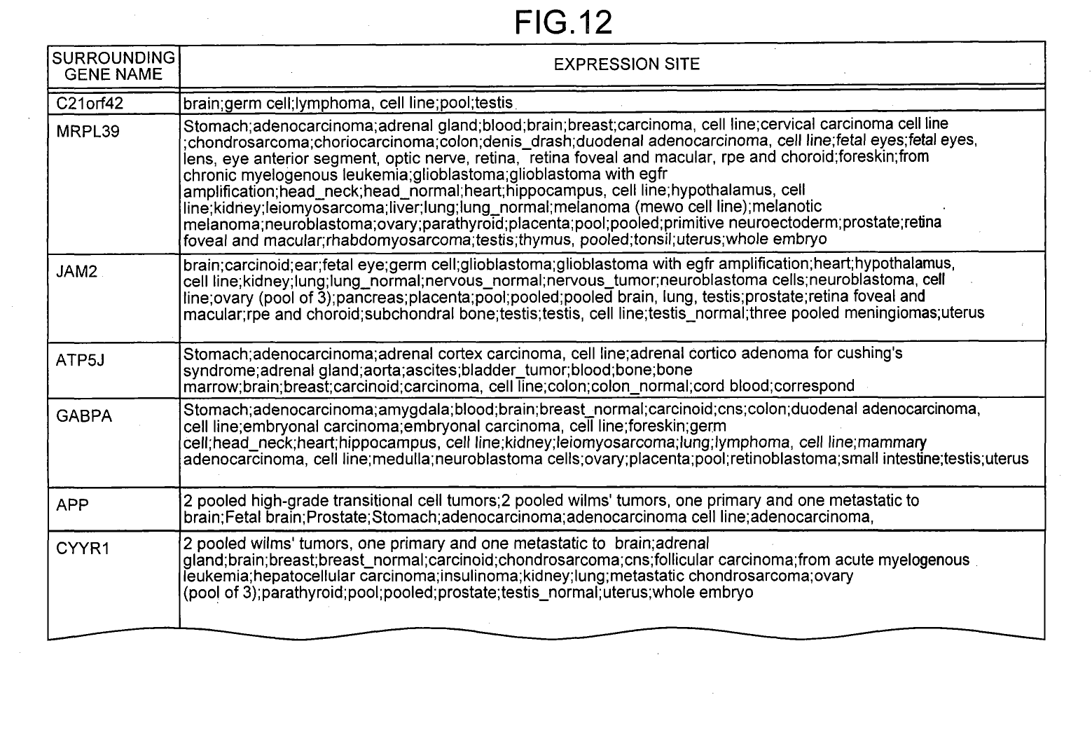

FIG. 12 is an example of expression information of the surrounding genes shown in FIG. 6, and the expression information indicates the result of extracting at Step S504. In FIG. 12, names of surrounding genes are described on the left column and contents of the expression sites on the right column. The surrounding genes are sorted in ascending order of the distance and described as similar to FIG. 10. They may be sorted at the time of output in Step S505.

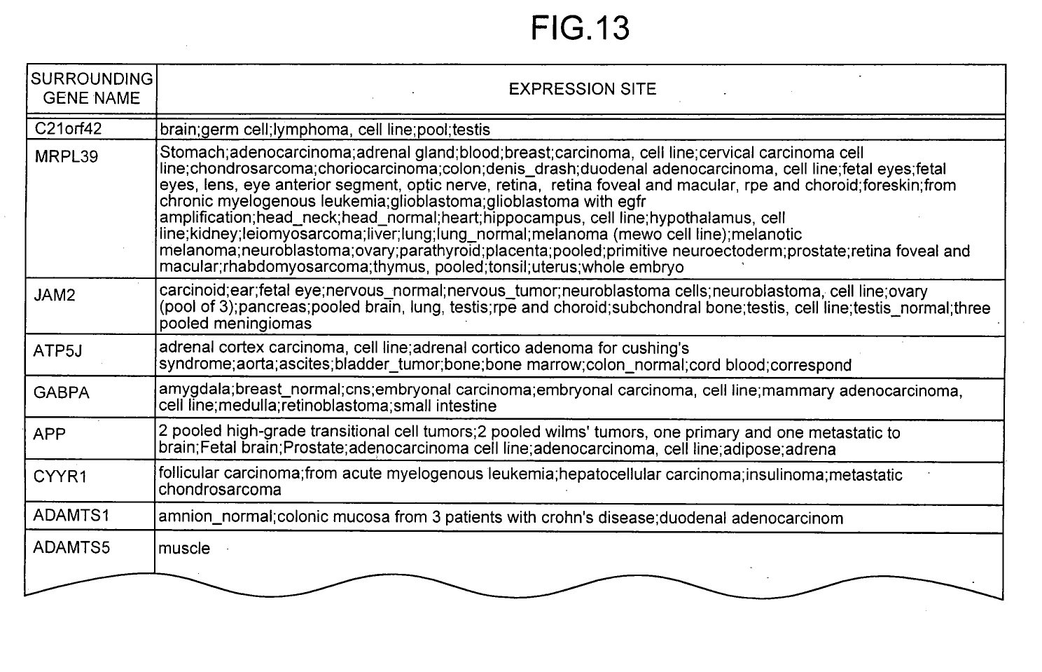

In the list shown in FIG. 12, expression sites in lower order are deleted from the list if they are the same as expression sites in higher order. For example, as shown in FIG. 12, the surrounding gene “MRPL39” located in lower order than the surrounding gene “C21orf42” in the most vicinity of the unknown gene has an expression tissue “testis”. Though, it is found that the surrounding gene “C21orf42” has already expressed at the expression tissue “testis”. Therefore, the expression site is deleted from the item of the lower surrounding gene “MRPL 39”. Such operations are repeatedly performed to the lowermost surrounding gene. A deleted result is shown in FIG. 13.

After Steps S501 to S505 are completed, the list is obtained as shown in FIG. 13 with respect to the sequence A of the unknown gene. From this list, an expression site of the sequence A of the unknown gene can be predicted easily.

According to the embodiment as described, from the sequence of the unknown gene, its expression sites can be predicted easily. This is effective to reduce experimental costs and computer resources. Therefore, the use of the present invention makes it possible to promptly perform rapidly functional analysis of unknown genes, and can greatly contribute the development of the gene engineering.

In the present embodiment, as shown in FIG. 2, the expression sites of the unknown gene 101 is predicted based on the trend in that a given gene expresses on the same tissue as its surrounding genes (genes 102, 103). Therefore, the longer the distance between the unknown gene 101 and the surrounding gene (genes 102, 103), the lower the precision of prediction drops. To maintain the precision of prediction, the distance cut off by a threshold is employed for predicting gene expression sites. The threshold is determined based on the sensitivity and specificity.

The sensitivity indicates a ratio of expression sites predicted (i.e. extracted) to expression sites previously determined that it is where the unknown gene expresses by another method. The higher the sensitivity, the less the pseudo-negative result arises and the higher the precision of prediction improves. The specificity indicates a ratio of expression sites not predicted to expression sites previously determined that it is where the unknown gene never expresses by another method. The higher the specificity, the less the pseudo-positive result arises and the higher the precision of prediction improves.

Therefore, based on the ratio (sensitivity) of expression sites extracted at Step S504 in FIG. 5, among expression sites previously determined by another method, on which the unknown gene 101 expresses, and on the ratio (specificity) of expression sites not extracted at Step S504, among expression sites previously determined by another method, on which the unknown gene never expresses, the threshold of the distance calculated at Step S503 is determined. Then, only the surrounding genes located within the determined threshold from target gene are to be sorted (Step S505). The Step S505 has the same process as described above.

Table 1 is a relationship between the sensitivity and the specificity.

TABLE 1

| | | Number of |
|---|---|---|
| | Number of Expression | Non-Expression sites |
| | sites of Unknown gene | of Unknown gene |
| | Determined by Another | Determined by |
| | Method | Another Method |
| Number of Expression | A | a |
| sites Predicted | | |
| Number of Expression | B | b |
| sites Not Predicted | | |

The sensitivity and the specificity can be derived from the following calculations:

Sensitivity=A/(A+B)

Specificity=b/(a+b)

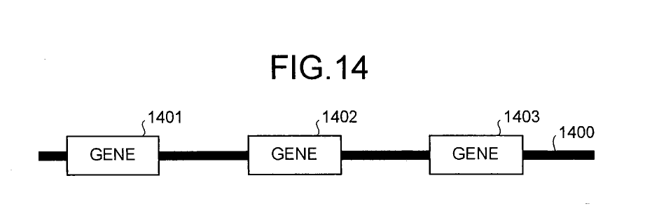

FIG. 14 is a diagram showing a positional relation of genes on a genome sequence, from which the sensitivity and the specificity are derived. Through the use of the information on the known gene, as shown in FIG. 14, genes 1401, 1402, and 1403 can be mapped onto a genome sequence 1400. To apply the predicting method to these genes, the gene 1401 is assumed as an unknown gene, and the genes 1402 and 1403 as surrounding genes. The same method is performed for the genes 1402 and 1403. Thresholds of the distance between the unknown gene and the surrounding gene are determined from, for example, an initial value of 100 kilo base-pair to 3000 kilo base-pair at an interval of 100 kilo base-pair. Averages of sensitivity and specificity calculated for each unknown gene within thresholds are calculated. Then, the calculated averages are plotted on a coordinate plane, which has the axis of abscissas indicating threshold between unknown and surrounding genes and the axis of ordinates indicating sensitivity/specificity.

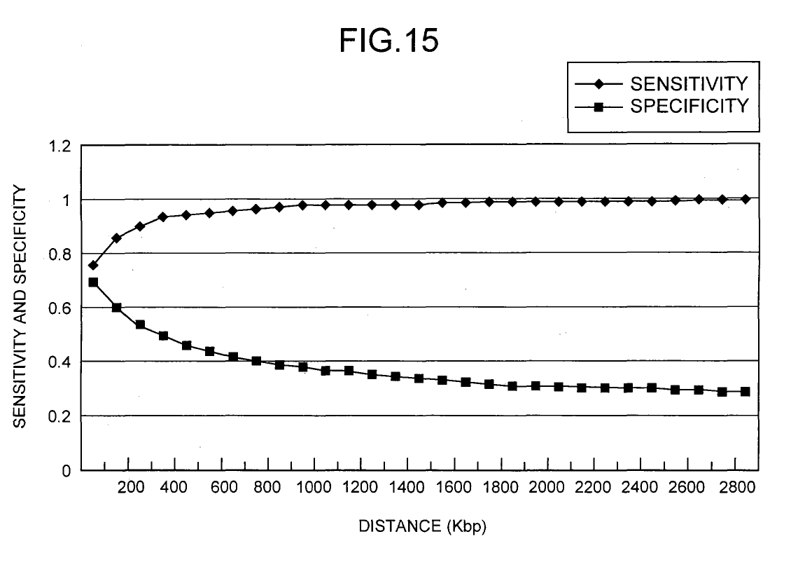

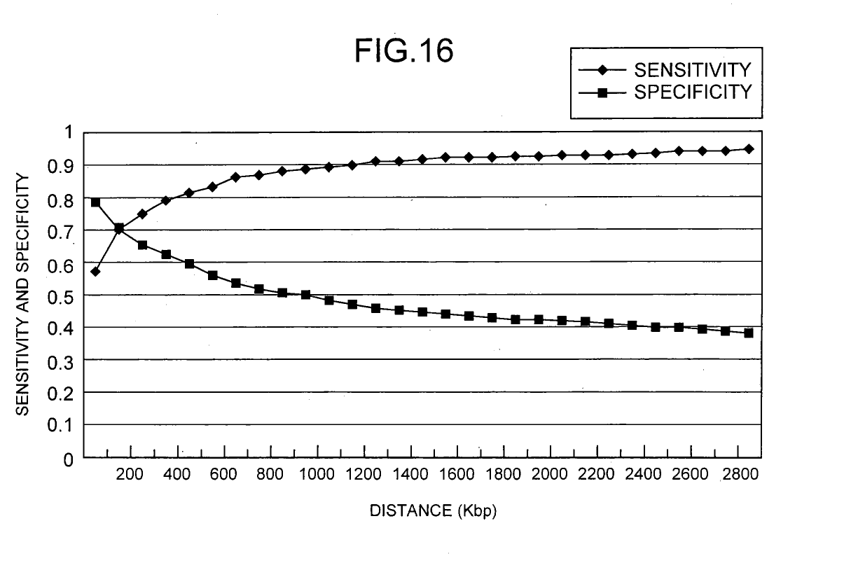

FIG. 15 is a diagram (graph) showing computational results using the known data associated with the human chromosome 19. FIG. 16 is a diagram (graph) showing computational results using the known data associated with the human chromosome 21. The axis of abscissas indicates threshold and the axis of abscissas indicates sensitivity/specificity both in FIGS. 15 and 16. In FIG. 15, the longer the distance between unknown and surrounding genes, the lower the specificity drops and the higher the sensitivity elevates to the contrary. When a cross-point between the specificity and the sensitivity is employed as a threshold, pseudo-negative and pseudo-positive results can be reduced. This is possibly effective to maintain the precision of prediction. A threshold in FIG. 15 is found on 100 kilo base-pair, for example. Similarly, a threshold in FIG. 16 is found on 200 kilo base-pair, for example.

Thus, the threshold depends on chromosome. Accordingly, it is desirable to find respective thresholds for all chromosomes before prediction through the above calculations. The following results show the predicted expression sites using the threshold derived from the sensitivity and specificity.

### EXAMPLE 1

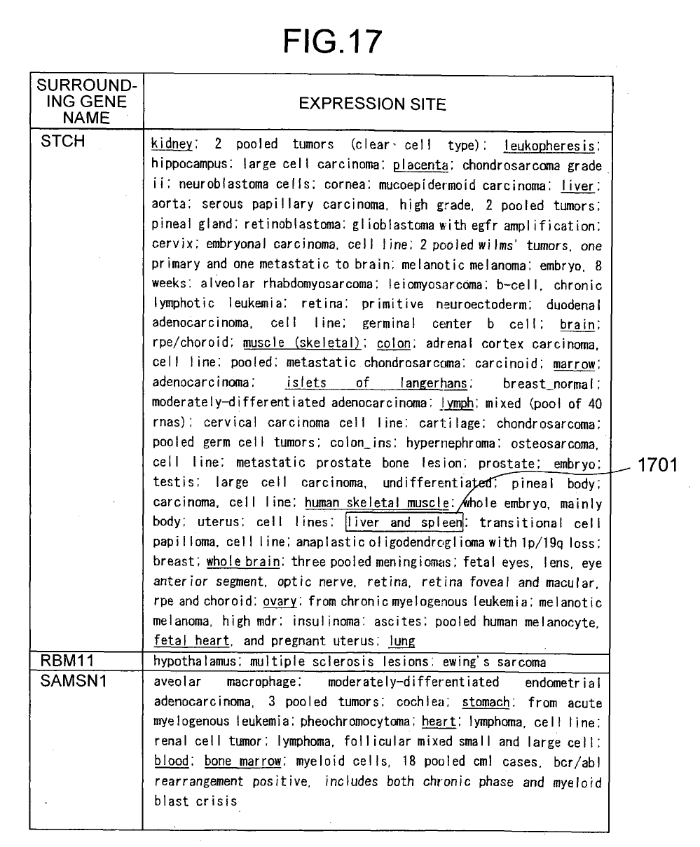

Human gene ABCC13 is employed to predict expression sites. FIG. 17 is a diagram showing the predicted results of the expression sites of the known gene (ABCC13). Human gene ABCC13, present on the human chromosome 19, is known in which its expression site is “Liver and Spleen” by the public database (UniGene).

On the other hand, as shown in FIG. 17, the surrounding gene “STCH” has an expression site “Liver and Spleen” denoted by reference number 1701, and this means that the expression site is correctly predicted. The paper (Biochem Biophys Res Commun. 2002 Dec. 6; 299 (3): 410-7) confirms the expression sites of the human gene ABCC13 on the expression sites underlined in FIG. 17.

### EXAMPLE 2

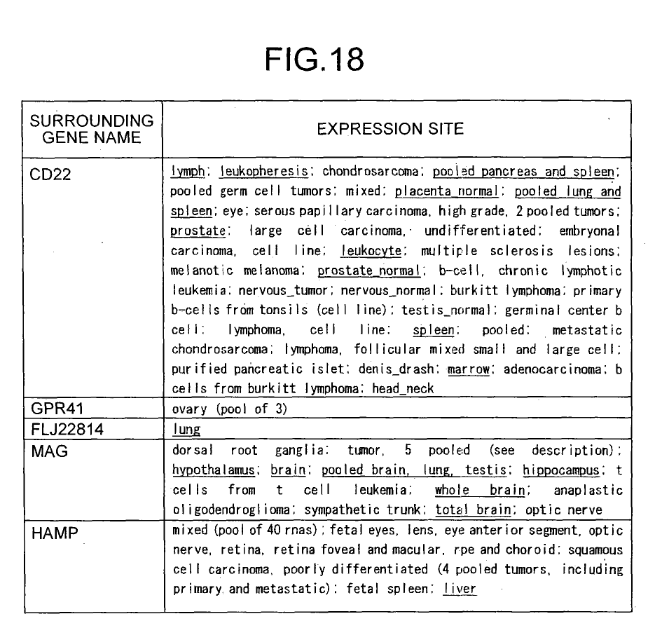

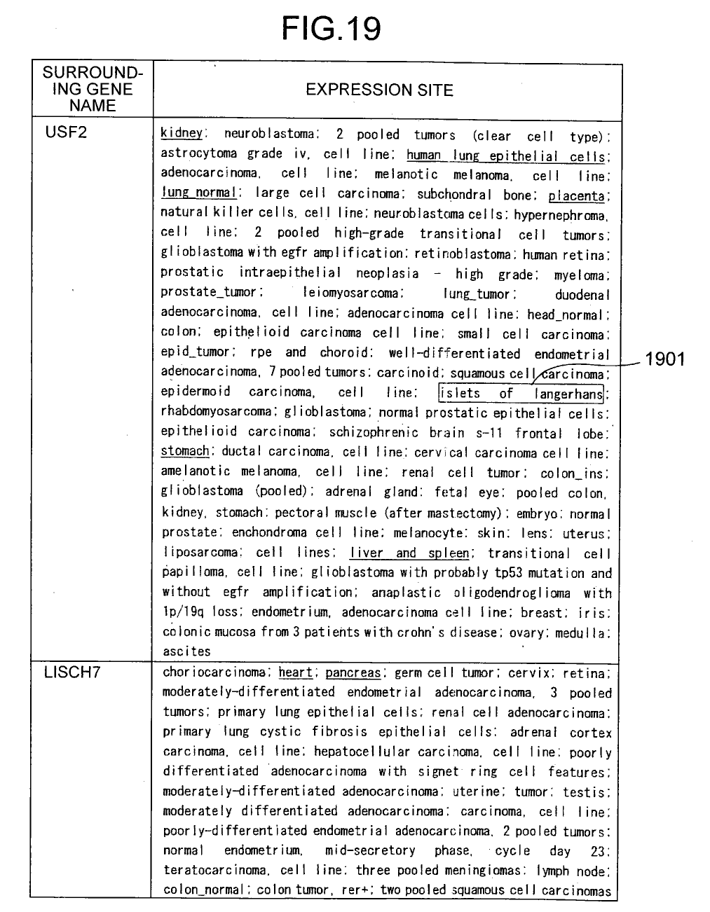

Human gene GPR40 is employed to predict expression sites. FIGS. 18 and 19 are diagrams showing the predicted results of the expression sites of another known gene (Human gene GPR40). Human gene GPR40, present on the human chromosome 21, is known in which its expression site is “Islets of Langerhans” in human pancreas by the public database (UniGene).

On the other hand, as shown in FIG. 19, the surrounding gene “USF2” has an expression site “Islets of Langerhans” denoted by reference number 1901, and this means that the expression site is correctly predicted. The paper (J Biol Chem. 2003 Mar. 28; 278 (13): 11303-11) confirms the expression sites of the human gene GPR40 on the expression sites underlined in FIGS. 18 and 19.

The method for predicting gene expression sites according to the embodiment may be achieved by a previously prepared computer-readable program, which is executed in computer such as a personal computer and a workstation. The program is stored in a computer-readable storage medium such as a HD, a FD, a CD-ROM, an MO and a DVD, and is read out of the storage medium by a computer to execute it. The program may include a transmission medium that can be delivered via a network such as the Internet.

As described above, genes on the genome sequence spaced by shorter distances from each other exhibit a trend to express on the same tissue. According to the invention utilizing this trend, it is possible to estimate an expression site of the unknown gene from information on the expression sites of the surrounding gene. This is effective to achieve a method, program and apparatus for predicting gene expression sites, which is possible to execute quick and efficient expression sites prediction and functional analysis for an unknown gene.

Although the invention has been described with respect to a specific embodiment for a complete and clear disclosure, the appended claims are not to be thus limited but are to be construed as embodying all modifications and alternative constructions that may occur to one skilled in the art which fairly fall within the basic teaching herein set forth.

What is claimed is:

1. A method for predicting gene expression sites, comprising:
calculating a distance between first and second genes on a genome sequence, wherein an expression site of the first gene is unknown, and the second gene is one of a plurality of known genes whose expression sites are known; and
determining the expression sites of the first gene based on the distance.

2. The method according to claim 1, wherein
the calculating includes calculating the distance for each of the plurality of genes, and
the determining includes determining the expression sites of the first gene as an expression site of at least one gene that has a predetermined distance relation among the plurality of genes.

3. The method according to claim 1, wherein
the calculating includes calculating a distance between the start position of the first gene and the start position of the second gene on the genome sequence.

4. The method according to claim 1, wherein
the calculating includes calculating a distance between the end position of the first gene and the end position of the second gene on the genome sequence.

5. The method according to claim 1, wherein
the calculating includes calculating a distance between the start position of the first gene and the end position of the second gene on the genome sequence.

6. The method according to claim 1, wherein
the calculating includes calculating a distance between the end position of the first gene and the start position of the second gene on the genome sequence.

7. The method according to claim 1, wherein
the calculating includes calculating a distance between first and second positions, the first position being between the start and end positions of the first gene on the genome sequence, and the second position being between the start and end positions of the second gene on the genome sequence.

8. The method according to claim 1, wherein
the calculating includes calculating a distance between a position between the start and end positions of the first gene and the start position of the second gene on the genome sequence.

9. The method according to claim 1, wherein
the calculating includes calculating a distance between a position between the start and end positions of the first gene and the end position of the second gene on the genome sequence.

10. The method according to claim 1, wherein
the calculating includes calculating a distance between the start position of the first gene and a position between the start and end positions of the second gene on the genome sequence.

11. The method according to claim 1, wherein
the calculating includes calculating a distance between the end position of the first gene and a position between the start and end positions of the second gene on the genome sequence.

12. A method for predicting gene expression sites, comprising:
inputting sequence information of a first gene whose expression sites are unknown;
acquiring a position of the first gene in a genome sequence associated with the sequence information;
searching for a second gene whose expression sites are known, the second gene being located around the position of the first gene on the genome sequence;
acquiring information of the expression sites of the second gene;
calculating a distance between the first gene and the second gene on the genome sequence;
sorting a plurality of second genes in ascending order of the distance; and
outputting the information of the expression sites of the second gene sorted at the sorting.

13. The method according to claim 12, wherein
the outputting includes outputting information of the expression sites which differs from that of the preceding second gene in the ascending order.

14. The method according to claim 12, further comprising:
displaying a list of the information of the expression sites of the second gene sorted at the sorting.

15. The method according to claim 12, wherein
the calculating includes calculating a distance between the start position of the first gene and the start position of the second gene on the genome sequence.

16. The method according to claim 12, wherein
the calculating includes calculating a distance between the end position of the first gene and the end position of the second gene on the genome sequence.

17. The method according to claim 12, wherein
the calculating includes calculating a distance between the start position of the first gene and the end position of the second gene on the genome sequence.

18. The method according to claim 12, wherein
the calculating includes calculating a distance between the end position of the first gene and the start position of the second gene on the genome sequence.

19. The method according to claim 12, wherein
the calculating includes calculating a distance between first and second positions, the first position being between the start and end positions of the first gene on the genome sequence, and the second position being between the start and end positions of the second gene on the genome sequence.

20. The method according to claim 12, wherein
the calculating includes calculating a distance between a position between the start and end positions of the first gene and the start position of the second gene on the genome sequence.

21. The method according to claim 12, wherein
the calculating includes calculating a distance between a position between the start and end positions of the first gene and the end position of the second gene on the genome sequence.

22. The method according to claim 12, wherein
the calculating includes calculating a distance between the start position of the first gene and a position between the start and end positions of the second gene on the genome sequence.

23. The method according to claim 12, wherein
the calculating includes calculating a distance between the end position of the first gene and a position between the start and end positions of the second gene on the genome sequence.

24. The method according to claim 12, wherein
the calculating includes determining a threshold of the distance, based on a first ratio of a first expression site to expression sites where the unknown gene expresses and a second ratio of a second expression site to expression sites where the unknown gene does not express, the first expression site is acquired by the acquiring of information of the expression sites, and the second expression site is not acquired by the acquiring of information of the expression sites.

25. A computer program product including computer executable instructions, for predicting gene expression sites, wherein the instructions, when executed by the computer, cause the computer to perform:
calculating a distance between first and second genes on a genome sequence, wherein an expression site of the first gene is unknown, and the second gene is one of a plurality of genes whose expression sites are known; and
determining the expression sites of the first gene based on the distance.

26. The computer program product according to claim 25, wherein
the determining include determining the expression sites of the first gene as an expression site of at least one gene that has a predetermined distance relation among the plurality of genes.

27. A computer program product including computer executable instructions, for predicting gene expression, wherein the instructions, when executed by the computer, cause the computer to perform:
inputting sequence information of a first gene whose expression site is unknown;
acquiring a position of the first gene in a genome sequence associated with the sequence information;
searching for a second gene whose expression sites are known, the second gene being located around the position of the first gene on the genome sequence;
acquiring information of the expression sites of the second gene;
calculating a distance between the first gene and the second gene on the genome sequence;
sorting a plurality of second genes in ascending order of the distance; and
outputting the information of the expression sites of the second gene sorted at the sorting.

28. An apparatus for predicting gene expression, comprising:
a calculation unit that calculates a distance between first and second genes on a genome sequence, wherein an expression site of the first gene is unknown, and the second gene is one of a plurality of genes whose expression sites are known; and
a determination unit that determines the expression sites of the first gene based on the distance.

29. The apparatus according to claim 28, wherein
the calculation unit calculates the distance for each of the plurality of genes, and
the determination unit determines the expression sites of the first gene as an expression site of at least one gene that has a predetermined distance relation among the plurality of genes.

30. An apparatus for predicting gene expression sites, comprising:
an input unit to input sequence information of a first gene whose expression site is unknown;
a positional information acquisition unit that acquires a position of the first gene in a genome sequence associated with the sequence information;
a searching unit that searches for a second gene whose expression sites are known, the second gene being located around the position of the first gene on the genome sequence;
an expression site information acquisition unit that acquires information of the expression sites of the second gene;
a calculation unit that calculates a distance between the first gene and the second gene on the genome sequence;
a storage unit that stores a plurality of second genes in ascending order of the distance; and
an output unit that outputs the information of the expression sites of the second gene sorted by the storage unit.
